(19) United States
(12) Patent Application Publication
(10) Pub. No.: US 2017/0056685 A1
(43) Pub. Date: Mar. 2, 2017

(54) SKIN CARE SYSTEM, DEVICE AND METHOD

(71) Applicant: KONINKLIJKE PHILIPS N.V., EINDHOVEN (NL)

(72) Inventors: SEVERIN LUC RAMSES HARVEY, EINDHOVEN (NL); MARJOLEIN YVONNE JANSEN, EINDHOVEN (NL); DIETER STURM, EINDHOVEN (NL); ADRIANUS THEODORUS HENDRIKUS SCHOENMAKERS, EINDHOVEN (NL)

(21) Appl. No.: 15/239,066

(22) Filed: Aug. 17, 2016

(30) Foreign Application Priority Data
Aug. 25, 2015 (EP) 15182251

(51) Int. Cl. A61N 5/06; A61H 23/00; A61B 5/00

(52) U.S. Cl. CPC A61N 5/0616; A61B 5/442; A61B 5/443; A61B 5/4836; A61H 23/00; A61N 2005/0644; A61N 2005/0626; A61H 2201/10

(57) ABSTRACT

The present invention relates to a skin care system (10), comprising: (i) a skin treatment device (12) for treating a skin of a user; (ii) a receiving unit (14) for receiving information on the skin of the user; and (iii) an evaluation unit (16) which is configured to evaluate the received information on the skin of the user and to determine based on said evaluation one or more personalized device settings for operating the skin treatment device (12). The skin treatment device (12) comprises a control unit (36) which is configured to operate the skin treatment device (12) upon initiation based on the determined one or more personalized device settings.

### FIELD OF THE INVENTION

The present invention relates to the field of skin care and cosmetics. In particular, the present invention relates to a skin care system for treating a skin of a user. Further aspects of the present invention relate to a corresponding method, a skin evaluation device, a skin treatment device as well as to a treatment head for such a skin treatment device.

### BACKGROUND OF THE INVENTION

In the field of skin care and cosmetics more and more products come into the market, especially skin care devices. Such skin care devices are designed for the general target audience. On the other hand, there is an increasing need to adjust the skin care devices to the user's skin condition, usage patterns and the environment. This is especially due to the fact that skin conditions and skin concerns are very individual factors that differ from person to person sometimes significantly. However, the trend of individualizing skin care devices makes it more complicated for users to choose the correct devices, and it makes it more complicated for users to handle the devices in a proper way.

Consumers usually have very little knowledge on their skin. Often they are not sure which treatment is the best for their skin. Hence, they do not know which products to choose. The result in such situations is that consumers rather often make use of products that are designed for average use and not designed for specific skin conditions and personal needs. Such “average” products, however, are less effective.

US 2015/0045631 A1 relates to an integrated system for skin and hair care that provides customized personal analysis of a user's specific situation and supplies consumer product recommendations to the user. The system thereto monitors measurable skin parameters, such as texture, wrinkles, radiance, skin physiology, skin biochemistry, etc. Based on these skin parameters the system then determines individualized advices that advise the users which skin care products to be used best for their skin.

Even though the system of US 2015/0045631 A1 already provides several benefits with respect to the above-mentioned problems, there is still room for improvement.

### SUMMARY OF THE INVENTION

It is an object of the present invention to provide a system, method and devices for treating the skin of a user in a manner that is individualized to the individual skin conditions of the user as good as possible. It is particularly an object of the present invention to facilitate the handling of skin care devices so as to increase consumer satisfaction and to help consumers to treat their skin in an easy but effective way.

In a first aspect of the present invention a skin care system is presented that comprises:

a skin treatment device for treating a skin of a user;

a receiving unit for receiving information on the skin of the user; and

an evaluation unit which is configured to evaluate the received information on the skin of the user and to determine based on said evaluation one or more personalized device settings for operating the skin treatment device;

wherein the skin treatment device comprises a control unit which is configured to operate the skin treatment device upon initiation based on the determined one or more personalized device settings.

In a second aspect of the present invention a method is presented that comprises the steps of:

providing a skin treatment device for treating a skin of a user, wherein the skin treatment device comprises a memory unit and a control unit that is configured to operate the skin treatment device upon initiation based on one or more personalized device settings stored in the memory unit;

receiving information on the skin of the user;

evaluating the received information on the skin of the user;

determining based on said evaluation the one or more personalized device settings for operating the skin treatment device; and

storing the determined one or more personalized device settings in the memory unit.

In a further aspect of the present invention a skin evaluation device is presented that comprises:

a receiving unit for receiving information on a skin of a user;

an evaluation unit which is configured to evaluate the received information on the skin of the user and to determine based on said evaluation one or more personalized device settings for operating a skin treatment device; and

a data transmission unit for transmitting the personalized device settings from the evaluation unit to the skin treatment device.

In a further aspect of the present invention a skin treatment device for treating a skin of a user is presented that comprises:

a memory unit having stored thereon one or more personalized device settings for operating the skin treatment device which have been determined based on an evaluation of information on the skin of the user; and

a control unit which is configured to operate the skin treatment device upon initiation based on the one or more personalized device settings stored in the memory unit.

In a still further aspect of the present invention a treatment head for a skin treatment device is presented for treating a skin of a user, the skin treatment device comprising (i) a base unit with a device housing, (ii) a driving unit which is arranged in the device housing and configured to drive the treatment head, and (iii) a control unit which is arranged in the device housing and configured to control the driving unit to drive the treatment head based on personalized device settings which have been determined based on an evaluation of information on the skin of the user, wherein the treatment head comprises a memory unit which is configured to store the personalized device settings, and wherein the treatment head is configured to be releasably coupled to the device housing.

Preferred embodiments of the invention are defined in the dependent claims. It shall be understood that the claimed method, the claimed skin evaluation device, the claimed skin treatment device, and the claimed treatment head have similar and/or identical preferred embodiments as the claimed skin care system and as defined in the dependent claims.

One of the main benefits of the herein presented system in contrast to the system proposed in US 2015/0045631 A1 is that the system according to the present invention does not only determine personalized advices that a user may follow or not, but directly determines based on an evaluation of the received skin information of the user one or more personalized device settings for operating a skin treatment device that is also part of the system. This provides the main advantage that the user does not have to adjust the settings of the skin treatment device him-/herself, which many consumers find difficult. Instead, the adjustment of the device settings is so to say automatized by the system and individualized for each consumer individually.

The system in a first step receives and evaluates information on the skin of the user. This information may comprise a plurality of skin parameters measured by means of one or more sensors. These one or more sensors may either be external sensors or sensors that are part of the system. The skin parameters measured by means of the one or more sensors may e.g. include (i) a parameter indicative of an amount of cutaneous sebum of the skin, (ii) a parameter indicative of a hydration state of the skin, (iii) a parameter indicative of an elasticity characteristic of the skin, (iv) a parameter indicative of an amount and/or characteristic of pores of the skin, (v) a parameter indicative of an amount and/or characteristic of wrinkles of the skin, (vi) a parameter indicative of an extent of UV-damage of the skin, (vii) a parameter indicative of a skin cleansing, (viii) a parameter indicative of a makeup quantity on the skin, (ix) a parameter indicative of a redness of the skin, (x) a parameter indicative of a colour of the skin, and/or (xi) a parameter indicative of a pigmentation of the skin.

The evaluation unit then determines based on a evaluation of the received skin parameters one or more personalized device settings that are individually adapted to the skin conditions of the user. These device settings may be used for operating the skin treatment device which may e.g. be implemented as a handheld device.

Said skin treatment device comprises a control unit which is configured to operate the skin treatment device upon initiation based on the determined one or more personalized device settings. This means that the user only has to initiate the skin treatment device, e.g. by operating an on/off button to turn on the device, wherein the skin treatment device will automatically be operated in the way determined before based on the received individual skin parameters of the user.

Despite the fact that the user does not have to adjust the device settings him-/herself, this also provides the advantage that the skin treatment device itself may be implemented in a rather simple manner with very few control buttons, in the best case only comprising an on/off button. This again provides a production cost advantage for the manufacturer and makes the skin treatment device less error-prone for the consumer.

Typical personalized device settings that may be determined in the above-mentioned way may comprise at least one of (i) a rotational speed with which a treatment head of the device is driven, (ii) a vibration frequency speed with which the treatment head of the device is driven, (iii) a movement direction along which the treatment device is driven, (iv) a treatment time which defines how long the treatment head of the device is driven per treatment session, (v) an underpressure level generated at the treatment head, (vi) a frequency and/or pattern of an underpressure generated at the treatment head, (vii) a light intensity of a light generated at the treatment head, and (viii) a frequency of a light generated at the treatment head.

According to an embodiment, the evaluation unit is configured to evaluate the received information on the skin of the user by individually scoring the plurality of skin parameters and ranking them in predefined ranking scales.

Different types of skin characteristics may be evaluated based on the plurality of measured skin parameters. These skin characteristics may include one or more skin conditions that may be indicated in specific numbers and metrics, such as skin hydration, skin elasticity, cutaneous sebum, etc. The skin characteristics may, however, also refer to skin concerns of the user that are harder to quantify, such as visual appearance, visibility of fine lines, etc.

The algorithm implemented in the evaluation unit nevertheless tries to score the plurality of skin parameters received from the sensors so as to quantify the different skin evaluation characteristics. The ranking scales may include numeric scales, discreet scales, and/or analogue scales. Ranking the skin parameters in predefined ranking scales such as low/medium/high or small/medium/large or few/medium/many keeps the evaluation result straightforward and understandable for the user. The quantification of the skin characteristics is one of the key steps to derive the personalized device settings.

According to a further embodiment, the evaluation unit is configured to determine based on said evaluation not only one or more personalized device settings, but also a personalized advice for the user in graphical, text and/or audible form, wherein the skin care system further comprises an output unit for outputting the personalized advice.

The result of the above-mentioned evaluation, i.e. the quantified skin characteristics, may thus also be used for generating personalized advices that help the user to understand and learn more about his/her skin and what type of treatments could be advisable for improving it. The personalized advices may e.g. include a graphic that shows the users their individual skin characteristics, e.g. split up into different types of categories (skin conditions and skin concerns).

Several ways of outputting the personal advice to the user are conceivable. The personalized advice may be given to the user in the form of a graph, such as a spider diagram, a bar graph etc. The personalized advice may also include written text, e.g. telling the user what type of treatment he/she should use, for what time and how often. The personalized advice may also include audible messages, e.g. how to use the skin treatment device and with what type of treatment heads. In summary, the personalized advice given to the user may include the usage of a specific product, a product feature, and/or user routines. User routines may include a recommended frequency of use of the skin treatment device, certain recommended days in the week the skin treatment device should be used, and/or at what specific times of the day the skin treatment device should be used. User routines may further include a recommended sequence explaining which facial regions should be treated and in which order (e.g. left check then forehead, then right cheek, nose, etc.). The personalized advice may furthermore include lifestyle advices/recommendations, such as a recommended amount of sleep, a recommended amount of water to drink, etc.

According to a further embodiment, the receiving unit comprises an input interface for receiving personal data of the user, wherein the evaluation unit is configured to evaluate not only the received information on the skin of the user, but also the personal data of the user in order to determine the one or more personalized device settings and/or the personalized advice.

Such personalized data may e.g. include answers to a predefined questionnaire that provide information on the gender, the age, dexterity, and/or habits of the user. The personal data can improve and detail the evaluation so as to provide the personalized device settings and/or the personalized advice in a more accurate and more individually personalized way.

According to a further embodiment, the skin care system comprises at least one computing device which includes the receiving unit and/or the evaluation unit, wherein the skin treatment device is implemented as a handheld device that is structurally separate from the at least one computing device. In this embodiment the presented system may thus comprise two or three devices.

In a first alternative the system comprises a first device implemented as a computing device including the receiving unit and the evaluation unit, and a second device represented by the skin treatment device. The one or more sensors for measuring the skin parameters of the user may be connected to the computing device by means of a wireless or hard-wired data connection. The system may furthermore comprise a data transmission unit for transmitting the personalized device settings from the evaluation unit implemented in the first device to the skin treatment device (representing the second device).

In a second alternative of this embodiment the system comprises three devices, a first computing device including the receiving unit, a second computing device including the evaluation unit, and the skin treatment device as third device. A practical example for the first two computing devices would be a smartphone or tablet PC as first computing device including the receiving unit and a stationary server or cloud server as second computing device including the evaluation unit. Data transmission between the first computing device and the second computing device as well as between the second computing device and the skin treatment device may be realized by means of hard-wired data transmission and/or wireless data transmission techniques (similar as in alternative 1).

In a still further alternative embodiment, the skin treatment device is implemented as a handheld device with a device housing, wherein the receiving unit and/or the evaluation unit are arranged within the device housing.

According to this embodiment the system in other words includes only one device, namely the skin treatment device which includes all of the above-mentioned units (receiving unit and evaluation unit). The one or more sensors for measuring the skin parameters may optionally also be included in the skin treatment device. Compared to the other, alternative solutions with external computing devices this solution provides the advantage of a very compact system with only one handheld device.

Independent of the above-mentioned alternative embodiments defining whether the receiving unit and/or the evaluation unit are spread over one or more devices or even included in the skin treatment device, the skin treatment device according to all of these embodiments preferably comprises a memory unit for storing the one or more personalized device settings.

According to a particularly preferred embodiment of the present invention the skin treatment device comprises: (i) a base unit with a device housing in which the control unit and a driving unit is arranged; and (ii) at least one treatment head that is configured to be releasably coupled to the device housing, wherein the control unit is configured to control the driving unit to drive the at least one treatment head based on the determined one or more personalized device settings stored in the memory unit, and wherein the memory unit is arranged at or within the at least one treatment head.

This embodiment provides the following advantages: Since the memory unit, which stores the personalized device settings determined based on the above-mentioned evaluation performed by the evaluation unit, is arranged within the treatment head of the skin treatment device, it is not the base unit or the whole skin treatment device that becomes personalized to the user, but the treatment head itself. This opens up the possibility that several users, e.g. a family, together use one and the same base unit of the skin treatment device and each user is assigned one or more personal treatment heads.

The one or more treatment heads of each user may be those that are determined to suit best for treating the skin concerns of the respective user. In other words, if the system comprises more than one treatment head, e.g. a first treatment head and a second treatment head which may be exchanged such that either the first or the second treatment head is coupled to the base unit, the evaluation unit is configured to determine (i) first personalized device settings and (ii) second personalized device settings, wherein the first treatment head comprises a first memory unit for storing the first personalized device settings, wherein the second treatment head comprises a second memory unit for storing the second personalized device settings, and wherein the control unit is configured to control the driving unit based on the first device settings stored in the first memory unit if the first treatment head is attached to the base unit, and to control the driving unit based on the second device settings stored in the second memory unit if the second treatment head is attached to the base unit.

The data transfer needed to transfer the device settings stored in the memory unit (which is included in the treatment head) to the control unit (which is included in the base unit) may again be based on a hard-wired data connection or a wireless data connection.

### BRIEF DESCRIPTION OF THE DRAWINGS

These and other aspects of the invention will be apparent from and elucidated with reference to the embodiments described hereinafter. In the following drawings

FIG. 1 shows a first embodiment of a skin care system according to the present invention;

FIG. 2 shows a second embodiment of the skin care system according to the present invention;

FIG. 3 schematically shows an embodiment of a skin treatment device according to the present invention;

FIG. 4 schematically illustrates in a block diagram a method according to an embodiment of the present invention;

FIG. 5 illustrates several examples of a questionnaire that may be evaluated in the skin care system according to the present invention;

FIG. 6 schematically illustrates a first example of an evaluation result provided by the skin care system according to the present invention;

FIG. 7 schematically illustrates a second example of an evaluation result provided by the skin care system according to the present invention;

FIG. 8 schematically illustrates an exemplary personalized advice provided by the skin care system according to the present invention; and

FIG. 9 schematically illustrates an exemplary personalized device setting provided by the skin care system according to the present invention.

### DETAILED DESCRIPTION OF THE INVENTION

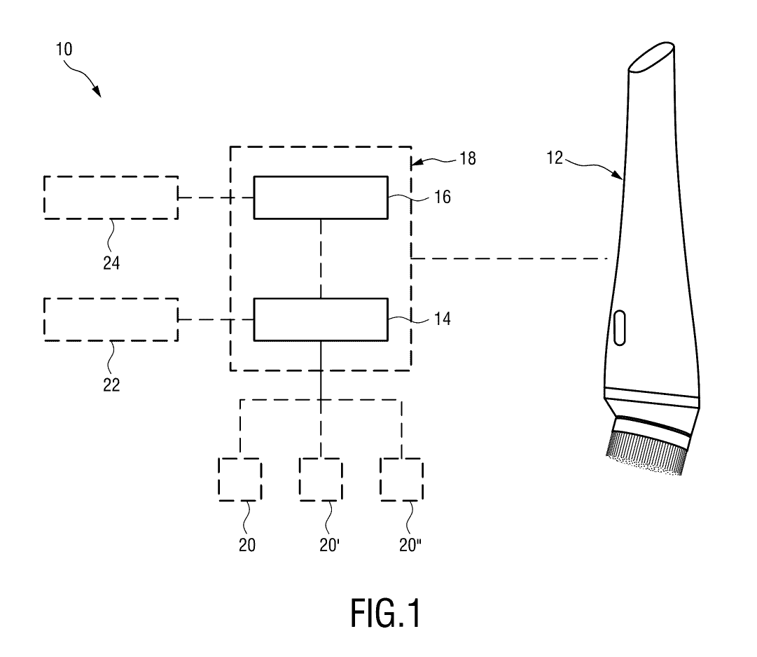

FIG. 1 schematically illustrates components of a skin care system according to an embodiment of the present invention. The skin care system is therein denoted in its entirety by reference numeral 10.

The skin care system 10 according to the first embodiment comprises a skin treatment device 12, a receiving unit 14, and an evaluation unit 16. The skin treatment device 12 is in this embodiment provided as a separate entity/device which is separate from the receiving unit 14 and the evaluation unit 16. The receiving unit 14 and the evaluation unit 16 may also be provided as separate entities/devices. However, in the exemplary embodiment that is schematically illustrated in FIG. 1 both the receiving unit 14 and the evaluation unit 16 are implemented in one and the same entity/device which shall be herein denoted as skin evaluation device 18.

The skin care system 10 may furthermore comprise one or more sensors 20, 20′, 20″, an input interface 22 and an output unit 24. The one or more sensors 20, 20′, 20″, the input interface 22 and the output unit 24 are, however, optional components of the system 10. These components may also be provided as external components that do not belong to the system 10 itself.

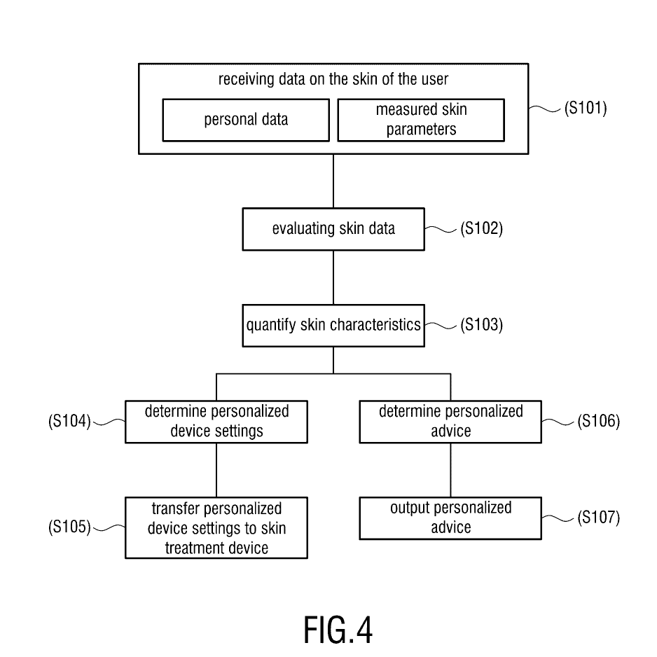

The function of the skin care system 10 may be explained with reference to the method which is schematically illustrated in FIG. 4.

The receiving unit 14 in a first step S101 receives information on the skin of a user. This information on the skin of the user (in the following simply denoted as skin data) preferably includes a plurality of skin parameters measured by means of the one or more sensors 20, 20′, 20″. Said measured skin parameters may e.g. include measurements of (a) cutaneous sebum, (b) hydration, (c) elasticity, (d) pores, (e) wrinkles, (0 pigmentation, (g) skin texture, (h) UV-damage, (i) skin cleansing, (j) makeup quantity on the skin, (k) redness of the skin, and/or (1) colour of the skin.

(a) Cutaneous sebum is a skin parameter that is indicative of how oily the skin is. This skin parameter is usually measured by means of a translusive measurement sensor.
(b) The hydration of the skin is preferably measured by means of a capacitive sensor.
(c) The elasticity of the skin is preferably measured by an especially designed skin elasticity measurement device that generates a vacuum for sucking the skin into a cavity for a certain distance, wherein the skin elasticity is determined by measuring the time the skin of the user needs to relax again (i.e. the time the skin needs to get back to its original shape).
(d) The number, structure and/or size of the pores is preferably measured by means of an optical sensor that captures an image of the skin and determines the number and size of the pores by means of an image analysis of the captured image.
(e) The wrinkles within the skin are measured in a similar way as the pores, i.e. by means of an optical sensor and a following image analysis.
(f)-(l) Pigmentation, UV-damage and the texture of the skin are preferably also derived from images taken of the face of the user. Makeup quantity may be e.g. measured by means of an optical or capacitive sensor. Redness and colour of the skin may be also measured by means of an optical sensor

In addition to the above-mentioned skin parameters measured by the one or more sensors 20, 20′, 20″ the receiving unit 14 may also receive personal data that are entered to the system 10 by means of the data input interface 22. The data input interface may e.g. be implemented as a keyboard, mouse or speech recognition sensor.

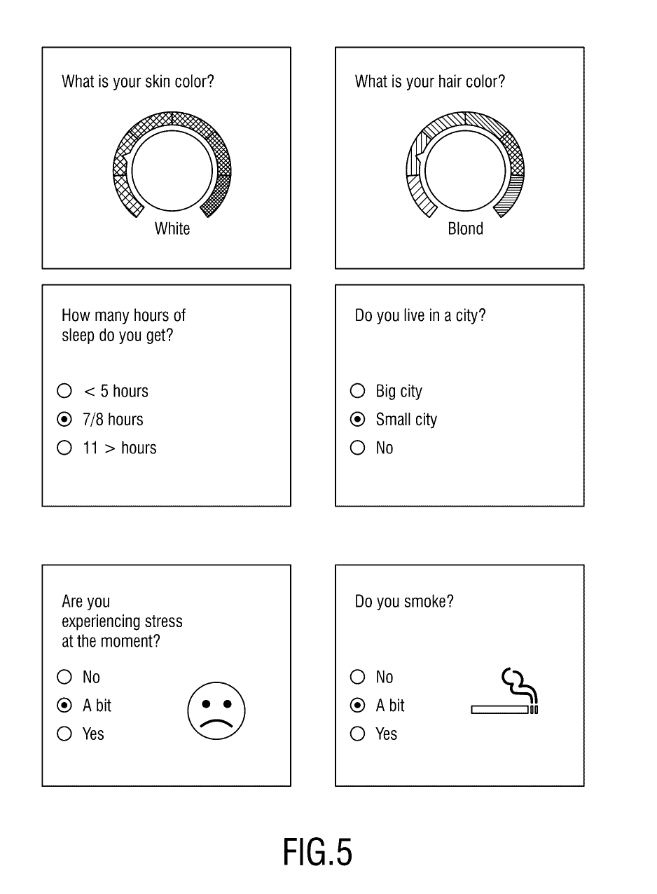

According to a preferred embodiment the personal data are entered to the system by providing a predefined questionnaire that the user may answer to provide his/her personal data. An exemplary excerpt of such a questionnaire is provided in FIG. 5. Personal data provided by the electronic questionnaire may e.g. include answers to questions regarding the skin color, the hair color, the amount of sleep, the environment of the user, the experienced stress level of the user, the smoking habits of the user, the gender and age of the user, the dexterity of the user, etc.

In the next step S102 the evaluation unit 16 evaluates the above-mentioned skin data. The evaluation unit thereto preferably carries out an algorithm that combines the above-mentioned different types of skin parameters and personal data to compute more detailed, quantified skin characteristics including skin conditions and skin concerns. This algorithm preferably scores the plurality of skin parameters and ranks them in predefined scales that are indicative of different types of skin characteristics.

Skin characteristics that may be used as evaluation criteria are (i) the number and extend of the pores, (ii) impurities of the skin, (iii) the radiance level of the skin, (iv) the pigmentation level of the skin, (v) the firmness of the skin, etc.

This quantifying/scoring/ranking of the skin characteristics in step S103 helps to make the received skin data of one user comparable to the skin data of another user. It also helps to give the users an objective feedback regarding the concerns and conditions of their skin, as this will be elucidated in more detail further below.

The evaluation unit 16 is furthermore configured to determine one or more personalized device settings based on the aforementioned evaluation and quantification of the skin data (step S104).

The personalized device settings determined by the evaluation unit 16 may then be used to operate the skin treatment device 12 in a manner that is specifically individualized for the user, i.e. specifically adapted to the individual skin characteristics of the user that have been analyzed and quantified before.

In the last step S105 the personalized device settings are transferred from the evaluation unit 16 to the skin treatment device 12. The skin treatment device 12 is in this step so to say programmed to the determined personalized device settings. The data transfer between the evaluation unit 16 and the skin treatment device 12 may be realized in many ways, either by means of a hard-wired data connection or by means of a wireless data connection.

The evaluation unit 16 may be furthermore configured to determine a personalized advice in addition to the personalized device settings (see step S106). This personalized advice may be output to the user in step S107 via the output unit 24 which is preferably implemented as a display.

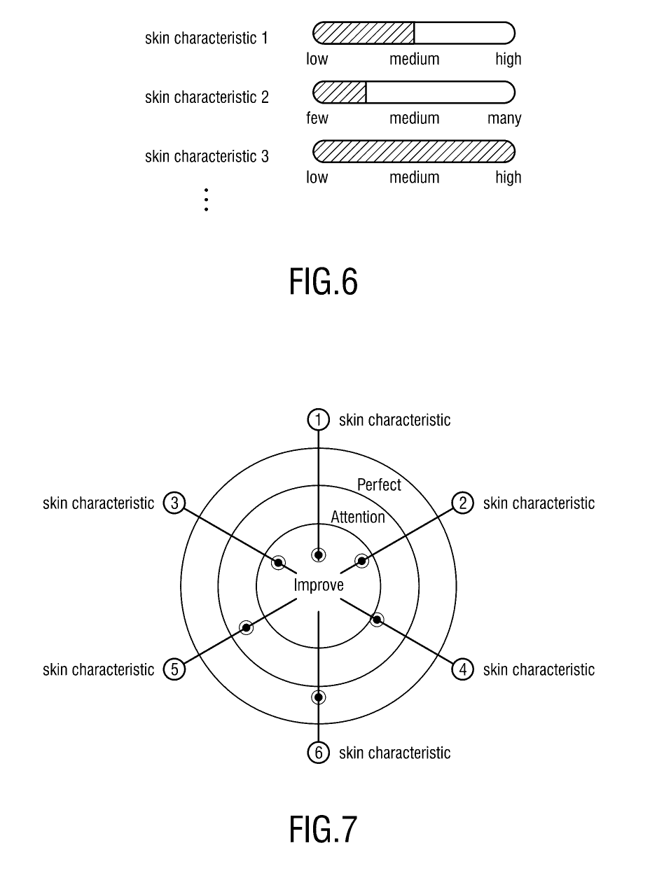

The personalized advice may on the one hand include the evaluation results visualized in graphical form. FIGS. 6 and 7 schematically illustrate two exemplary graphical representations that should help the user understanding more about his/her individual skin characteristics. These graphical representations are preferred to be kept as simple as possible in order to make it comprehensible for the user. The quantification of each skin characteristic/category may e.g. be shown in simple bar graphs as schematically illustrated in FIG. 6 or in a common spider graph as schematically illustrated in FIG. 7.

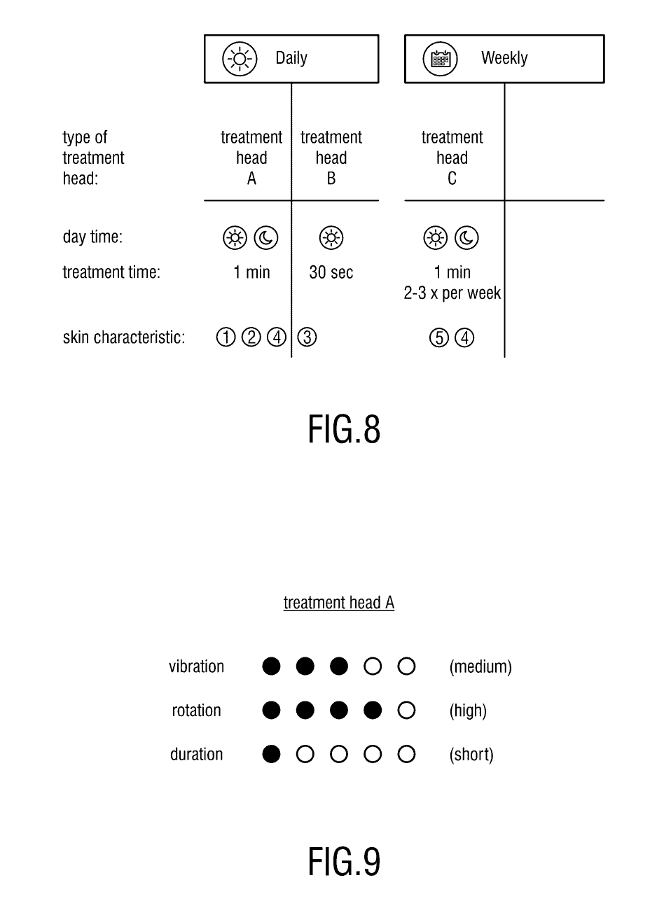

On the other hand, the personalized advice may include specific advices relating to which type of treatment head of the skin treatment device 12 the user should use, at what time, for how long, and/or to treat which skin characteristic. An example of such a specific personal advice is schematically illustrated in FIG. 8. The personal advice may finally also include information regarding the personalized device settings. As schematically illustrated in FIG. 9, such personalized device settings may include a vibration frequency, a rotation speed, a duration of the treatment, an underpressure level generated at the treatment head, a frequency and/or pattern of an underpressure generated at the treatment head, a light intensity of a light generated at the treatment head, and a frequency of a light generated at the treatment head. The type of device settings mainly depends from the type of treatment head used. The skin treatment device may be used with several types of treatment heads, e.g. brushes, massage heads, microdermabrasive treatment heads, light treatment heads, etc. The device settings may either be feedback to the user in text form, or, as shown in FIG. 9 in graphical form which is in most of the cases better comprehensible for the user.

However, it shall be noted that the personalized device settings do not necessarily have to be shown to the user, since they are anyway transferred to the skin treatment device 12 in the way explained above, such that the skin treatment device 12 is automatically operated based on these device settings upon initiation of the device 12. According to a preferred embodiment the skin treatment device 12 is configured to automatically carry out the operation based on the personalized device settings when being turned on (e.g. by pressing an on/off button).

Even though the general concept of the herein presented method has been described above with respect to FIG. 4 in a way that could imply a single algorithm that is carried out by the evaluation unit 16, one could also think of implementing three different algorithms: 1.) one algorithm for evaluating the measured skin parameters (and/or the personal data) and determining the personal skin characteristics of the user, 2.) another algorithm for translating the skin characteristics into the personalized device settings, and 3.) a third algorithm for translating the skin characteristics into the personalized user advice.

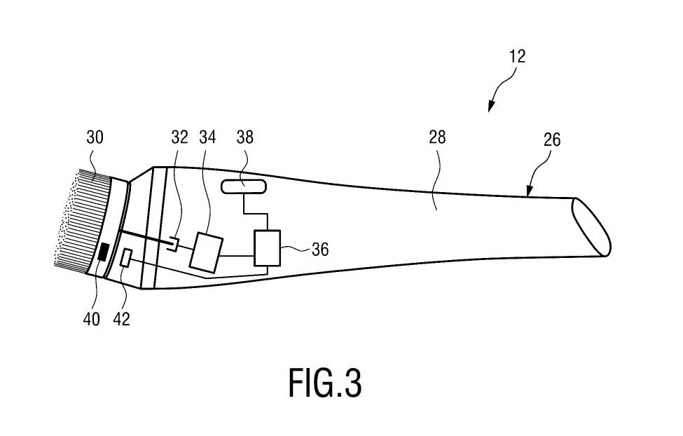

FIG. 3 schematically shows an embodiment of the skin treatment device 12. The skin treatment device 12 is preferably implemented as an electronic device that is either powered by main supplied electricity or battery-driven. The skin treatment device 12 is in general preferably configured to provide cosmetic treatments of the skin. It is preferably configured for cleaning and/or massaging the skin of the user. It may also be configured for other cosmetic treatments, such as thermal skin treatments or microdermabrasive skin treatments.

The skin treatment device 12 according to the embodiment illustrated in FIG. 3 comprises a base unit 26 which incorporates several components of the device 12 enclosed in a device housing 28. The skin treatment device 12 is preferably implemented as a modular device in the sense that different types of treatment heads 30 may be removably coupled to the base unit 26. The base unit 26 thereto preferably comprises a mechanical connector 32 for removably connecting the treatment head 30 to the device housing 28.

The treatment head 30 exemplarily shown in FIG. 3 comprises a brush for cleaning the skin of the user. The skin treatment device 12 furthermore comprises a driving unit 34 (e.g. an electro motor) that is configured to drive the treatment head 30, a control unit 36 (a controller or microprocessor) that is configured to control the driving unit 34, and an operating button 38 (on/off button) for operating/initiating the control unit 36.

The control unit 36 controls the driving unit 34 based on the above-mentioned personalized device settings that have been determined by the evaluation unit 16 and transferred to the skin treatment device 12. The skin treatment device 12 thereto comprises a memory unit 40 in which the personalized device settings are stored. This memory unit 40 may be arranged within the base unit 26. However, according to the embodiment shown in FIG. 3 it is arranged at or within the treatment head 30. The advantage of this configuration is that by transferring the determined personalized device settings to the memory unit 40 the treatment head 30 gets personalized (i.e. adapted to the specific needs of the user) and not the base unit 26. A plurality of users may thus use one and the same base unit 26 of the skin treatment device 12 together, while each user has his own one or more treatment heads 30 that are adapted and programmed to the individual needs of each user.

The base unit 26 according to this embodiment therefore comprises a data read-out unit 42 that is configured to read out the memory unit 40 and transfer the read-out device settings to the control unit 36. It shall be noted that the read-out device settings do not necessarily have to be directly transferred to the control unit 36, but may also be temporarily stored in a temporary memory unit (e.g. a buffer memory) that is implemented in the base unit 26.

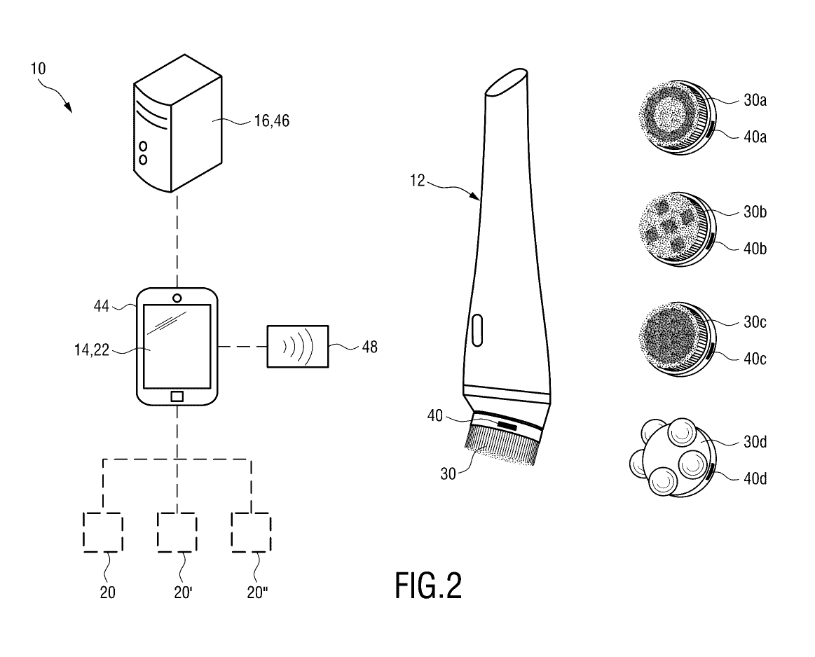

As it is illustrated in the embodiment shown in FIG. 2, the skin treatment device 12 may not only comprise a single type of treatment head 30, but a plurality of different types of treatment heads 30a-30d that are all directed and configured for specific types of skin treatments. This may, for example, include several types of brushes, such as a sensitive brush 30a, a deep pore brush 30b, and an acne brush 30c. A treatment head 30d which is especially designed for massaging the skin may also be attached to the base unit 26.

All these different types of treatment heads 30a-30d may be personalized to the user in the above-mentioned way by storing the personalized device settings that have been evaluated by the evaluation unit 16 in the memory unit 40a-40d of the respective treatment head 30a-30d. Each of the memory units 40a-40d may e.g. comprise at least two memory locations, one for storing the personalized device settings and the other one for storing a treatment head ID that identifies the type of treatment head. When attaching the treatment head 30 to the base unit 26 of the skin treatment device 12 the control unit 36 may thus not only implement the device settings stored in the respective memory unit 40a-40d, but also identify the type of treatment head 30a-30d that is attached to the base unit 26.

Further details of the skin care system 10 according to the second embodiment shown in FIG. 2 relate to the type of implementation of the receiving unit 14 and the evaluation unit 16. The receiving unit 14 is according to the second embodiment preferably implemented as a computing device 44, e.g. a smartphone, tablet PC, laptop or desktop PC. It may thus also comprise the input interface 22 for receiving the personal data (e.g. realized as a keyboard or touchscreen). The one or more sensors 20 may again be realized as external sensors that are connected to the computing device 44 by means of a hard-wired or wireless data connection.

The evaluation unit 16 is in contrast to the first embodiment in this case realized as a separate entity. The evaluation unit 16 may e.g. be implemented as a remote server or cloud server 46 which is accessible by the computing device 44 containing the receiving unit 14 via a data network, e.g. the internet.

In a practical example the user or another person would thus first collect the skin data by means of the one or more sensors 20, 20′, 20″ and the user or an assisting person could answer the personal questions of the above-mentioned predefined questionnaire that is either implemented on the computing device 44 or received from an online server or web platform. These skin data would then be transferred to the remote server 46 in order to be evaluated and quantified in the way explained above. The evaluation results will then be automatically sent back to the computing device 44, or they may be manually retrieved from the server 46 by means of the computing device 44 such that they may be shown to the user via the display of the computing device 44.

In order to personalize the different types of treatment heads 30a-30d of the user, a data transmission unit 48 could be used that is connected to the computing device 44 or even integrated therein. The data transmission unit 48 may e.g. be implemented as an NFC interface. The personalized device settings could then be transferred to the memory units 40a-40d by bringing the treatment heads 30a-30d into close proximity of the NFC interface. However, it shall be noted that other types of data transmission techniques, such as Bluetooth® or WLAN could be used alternatively.

Even though this is not specifically illustrated in the drawings, it is also conceivable to integrate all components of the system, especially the receiving unit 14 and the evaluation unit 16 (but optionally also the one or more sensors 20, 20′, 20″) in the skin treatment device 12 so as to be arranged within the device housing 28 of the base unit 26 of the device 12.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A skin care system (10), comprising:
a skin treatment device (12) for treating a skin of a user;
a receiving unit (14) for receiving information on the skin of the user; and
an evaluation unit (16) which is configured to evaluate the received information on the skin of the user and to determine based on said evaluation one or more personalized device settings for operating the skin treatment device (12);
wherein the skin treatment device (12) comprises a control unit (36) which is configured to operate the skin treatment device (12) upon initiation based on the determined one or more personalized device settings.

2. The skin care system of claim 1, wherein said information on the skin of the user comprises a plurality of skin parameters measured by means of one or more sensors (20), wherein said skin parameters include at least one of: (i) a parameter indicative of an amount of cutaneous sebum of the skin, (ii) a parameter indicative of a hydration state of the skin, (iii) a parameter indicative of an elasticity characteristic of the skin, (iv) a parameter indicative of an amount and/or characteristic of pores of the skin, (v) a parameter indicative of an amount and/or characteristic of wrinkles of the skin, (vi) a parameter indicative of an extent of UV-damage of the skin, (vii) a parameter indicative of a skin cleansing, (viii) a parameter indicative of a makeup quantity on the skin, (ix) a parameter indicative of a redness of the skin, (x) a parameter indicative of a colour of the skin, and (xi) a parameter indicative of a pigmentation of the skin.

3. The skin care system of claim 1, wherein the evaluation unit (16) is configured to evaluate the received information on the skin of the user by individually scoring the plurality of skin parameters and ranking them in predefined ranking scales.

4. The skin care system of claim 1, wherein the evaluation unit (16) is configured to determine based on said evaluation not only one or more personalized device settings, but also a personalized advice for the user in graphical, text and/or audible form, wherein the skin care system further comprises an output unit for outputting the personalized advice.

5. The skin care system of claim 1, wherein the receiving unit (14) comprises an input interface (22) for receiving personal data of the user, and wherein the evaluation unit (16) is configured to evaluate not only the received information on the skin of the user, but also the personal data of the user in order to determine the one or more personalized device settings.

6. The skin care system of claim 1, comprising at least one computing device (44, 46) which includes the receiving unit (14) and/or the evaluation unit (16), wherein the skin treatment device (12) is implemented as a handheld device that is structurally separate from the at least one computing device (44, 46).

7. The skin care system of claim 6, further comprising a data transmission unit (48) for transmitting the personalized device settings from the evaluation unit (16) to the skin treatment device (12).

8. The skin care system of claim 1, wherein the skin treatment device (12) is implemented as a handheld device with a device housing (28), wherein the receiving unit (14) and/or the evaluation unit (16) are arranged within the device housing (28).

9. The skin care system of claim 1, wherein the skin treatment device (12) comprises a memory unit (40) for storing the one or more personalized device settings.

10. The skin care system of claim 9, wherein the skin treatment device (12) comprises:
a base unit (26) with a device housing (28) in which the control unit (36) and a driving unit (34) is arranged; and
at least one treatment head (30) that is configured to be releasably coupled to the device housing (28),
wherein the control unit (36) is configured to control the driving unit (34) to drive the at least one treatment head (30) based on the determined one or more personalized device settings stored in the memory unit (40).

11. The skin care system of claim 10, wherein the memory unit (40) is arranged at or within the at least one treatment head (30).

12. The skin care system of claim 10, wherein the one or more personalized device settings comprises at least one of (i) a rotational speed with which the at least one treatment head is driven, (ii) a vibration frequency speed with which the at least one treatment head is driven, (iii) a movement direction along which the at least one treatment head is driven, (iv) a treatment time which defines how long the at least one treatment head is driven per treatment session, (v) an underpressure level generated at the treatment head, (vi) a frequency and/or pattern of an underpressure generated at the treatment head, (vii) a light intensity of a light generated at the treatment head, and (viii) a frequency of a light generated at the treatment head.

13. The skin care system of claim 11, wherein the at least one treatment head (30) includes a first treatment head (30a) and a second treatment head (30b) which may be exchanged such that either the first or the second treatment head (30a, 30b) is coupled to the base unit (26), wherein the evaluation unit (16) is configured to determine based on said evaluation (i) first personalized device settings and (ii) second personalized device settings, wherein the first treatment head (30a) comprises a first memory unit (40a) for storing the first personalized device settings, wherein the second treatment head (30b) comprises a second memory unit (40b) for storing the second personalized device settings, and wherein the control unit (36) is configured to control the driving unit (34) based on the first device settings stored in the first memory unit (40a) if the first treatment head (30a) is attached to the base unit (26), and to control the driving unit (34) based on the second device settings stored in the second memory unit (40b) if the second treatment head (30b) is attached to the base unit (26).

14. A method, comprising the steps of:
providing a skin treatment device (12) for treating a skin of a user, wherein the skin treatment device (12) comprises a memory unit (40) and a control unit (36) that is configured to operate the skin treatment device (12) upon initiation based on one or more personalized device settings stored in the memory unit (40);
receiving information on the skin of the user;
evaluating the received information on the skin of the user;
determining based on said evaluation the one or more personalized device settings for operating the skin treatment device (12); and
storing the determined one or more personalized device settings in the memory unit (40).

15. A skin evaluation device (18), comprising:
a receiving unit (14) for receiving information on a skin of a user;
an evaluation unit (16) which is configured to evaluate the received information on the skin of the user and to determine based on said evaluation one or more personalized device settings for operating a skin treatment device (12); and
a data transmission unit (48) for transmitting the personalized device settings from the evaluation unit (16) to the skin treatment device (12).

16. A skin treatment device (12) for treating a skin of a user, comprising:
a memory unit (40) having stored thereon one or more personalized device settings for operating the skin treatment device (12) which have been determined based on an evaluation of information on the skin of the user; and
a control unit (36) which is configured to operate the skin treatment device (12) upon initiation based on the one or more personalized device settings stored in the memory unit (40).

17. A treatment head (30) for a skin treatment device (12) for treating a skin of a user, the skin treatment device (12) comprising (i) a base unit (26) with a device housing (28), (ii) a driving unit (34) which is arranged in the device housing (28) and configured to drive the treatment head (30), and (iii) a control unit (36) which is arranged in the device housing (28) and configured to control the driving unit (34) to drive the treatment head (30) based on personalized device settings which have been determined based on an evaluation of information on the skin of the user,
wherein the treatment head (30) comprises a memory unit (40) which is configured to store the personalized device settings, and wherein the treatment head (30) is configured to be releasably coupled to the device housing (28).
